(19) United States
(12) Patent Application Publication
(10) Pub. No.: US 2021/0338863 A1
(43) Pub. Date: Nov. 4, 2021

(54) DECONTAMINATION APPARATUS

(71) Applicant: TESER TECHNOLOGIES INC., CALGARY (CA)

(72) Inventors: KAMAL HAMMAD, CALGARY (CA); MARK LEFSRUD, HUDSON (CA); PHILLIP ALLE, CALGARY (CA); BRETT KOSTKA, CALGARY (CA); JOHN FOX, AIRDRIE (CA)

(73) Assignee: TESER TECHNOLOGIES INC., CALGARY (CA)

(21) Appl. No.: 17/242,566

(22) Filed: Apr. 28, 2021

Related U.S. Application Data
(60) Provisional application No. 63/017,203, filed on Apr. 29, 2020.

(51) Int. Cl. A61L 2/10

(52) U.S. Cl. CPC A61L 2/10; A61L 2202/11; A61L 2202/121; C08L 27/18; A61L 2202/23; A61L 2202/24; A61L 2202/26; A61L 2202/122

(57) ABSTRACT

A decontamination apparatus includes a housing defining an interior volume and having at least a top, a bottom, a left side, a right side, a front, and a rear. The top, the bottom, the left and right sides can each have a set of low-voltage UV-C light emitting diodes that face towards the interior volume. The front can include a front opening and the rear can include a rear opening. The apparatus can include a front door that is openable and closable to block the front opening, and a rear door that is openable and closable to block the rear opening, to allow objects to pass through the interior volume from the front to the rear. Alternatively, a belt conveyor system can pass through the interior volume from outboard of the front opening to outboard of the rear opening to allow objects to be conveyed through the interior volume.

### CROSS-REFERENCES TO RELATED APPLICATIONS

This application claims the benefit of U.S. Provisional Patent Application No. 63/017,203 filed on Apr. 29, 2020, which is incorporated herein by reference in its entirety.

### FIELD

This document relates to decontamination. More specifically, this document relates to apparatuses for decontamination of objects, such as objects in a hospital setting.

### BACKGROUND

U.S. Pat. No. 8,203,124 (Havens et al.) discloses a sterilization apparatus and methods of using a sterilization system. The sterilization apparatus can take the form of a sterilization chamber comprising a top wall, a bottom wall, end walls and side walls which define the interior of the chamber. Throughout the interior of the chamber, multiple ultraviolet light emitting diodes irradiate energy at wavelengths for destroying pathogenic substances and achieving an efficient level of sterilization. A UV transparent plate located within the interior of the chamber can support one or more target devices, and can be proportionally sized to accommodate placement of a target device for the simultaneous and uniform distribution of UV sterilizing energy to the surfaces of a target. One or more interior surfaces of the chamber may include a UV reflective material for distributing UV radiation upon substantially all surfaces of a target.

### SUMMARY

The following summary is intended to introduce the reader to various aspects of the detailed description, but not to define or delimit any invention.

Decontamination apparatuses are disclosed.

According to some aspects, a decontamination apparatus includes a housing defining an interior volume and having at least a top, a bottom, a left side, a right side, a front, and a rear. At least one of the top, the bottom, the left side, and the right side includes a UV-C light source that faces inwardly towards the interior volume. The front includes a front opening and a front door that is openable and closable to block the front opening. The rear includes a rear opening and a rear door that is openable and closable to block the rear opening, to allow objects to pass through the interior volume from the front to the rear.

According to some aspects, a decontamination apparatus includes a housing defining an interior volume and having at least a top, a bottom, a left side, a right side, a front, and a rear. At least one of the top, the bottom, the left side, and the right side includes a UV-C light source that faces inwardly towards the interior volume. The front includes a front opening, and the rear includes a rear opening. A belt conveyor system passes through the interior volume from outboard of the front opening to outboard of the rear opening to allow objects to be conveyed through the interior volume.

According to some aspects, a decontamination apparatus includes a housing defining an interior volume and having a top, a bottom, a left side, a right side, a front, and a rear. Each of the top, the bottom, the left side, and the right side includes, respectively, a set of UV-C light emitting diodes (LEDs) that face towards the interior volume, a UV-C transmissive panel between the set of UV-C LEDs and the interior volume, and an inner wall including a support panel that supports the set of UV-C LEDs and that defines a recess in which the set of UV-C LEDs are nested.

According to some aspects, a decontamination apparatus includes a housing defining an interior volume and having at least a top, a bottom, a left side, a right side, a front, and a rear. At least one of the top, the bottom, the left side, the right side, the front, and the rear includes a set of low-voltage UV-C light emitting diodes (LEDs) that face inwardly towards the interior volume. A power adaptor is provided for connecting the low-voltage UV-C LEDs to a power source.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1 is a perspective view of an example decontamination apparatus, showing front and rear doors thereof in a closed configuration;

FIG. 2 is a front view of the decontamination apparatus of FIG. 1;

FIG. 3 is a perspective view of the decontamination apparatus of FIG. 1, showing front and rear doors thereof in an open configuration;

FIG. 4 is a cross-section taken along line 4-4 in FIG. 2;

FIG. 5 is a cross-section taken along line 5-5 in FIG. 2;

FIG. 6 is an exploded view of the decontamination apparatus of FIG. 1;

FIG. 7 is a perspective view of another example decontamination apparatus;

FIG. 8 is a perspective view of another example decontamination apparatus, showing a front door thereof in an open configuration; and

FIG. 9 is a perspective view of the decontamination apparatus of FIG. 8, showing the front door in a closed configuration.

### DETAILED DESCRIPTION

Various apparatuses or processes or compositions will be described below to provide an example of an embodiment of the claimed subject matter. No embodiment described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Generally disclosed herein are apparatuses that can be used for decontamination of various objects. As used herein, the term “decontamination” can refer to the reduction of microorganisms (e.g. viruses, bacteria, and fungi) on the surface of an object (i.e. disinfection), or the killing of all microorganisms on the surface of an object (i.e. sterilization). The apparatuses can be used to decontaminate various objects, such as but not limited to electronics (e.g. mobile telephones, tablets, and laptop computers), food (e.g. fruits and vegetables), clothing and wearable objects (e.g. personal protective equipment such as masks, gowns, and face shields), medical devices (e.g. surgical tools, thermometers, stethoscopes, blood pressure monitors, hemostats, and scissors), hospital equipment (e.g. ventilator components, carts, and stretchers), household and personal objects (e.g. keys), office supplies (e.g. pens), grooming tools (e.g. hair-cutting tools, nail-cutting tools, and tweezers), and cosmetics and applicators (e.g. makeup brushes). The apparatuses can decontaminate objects with the use of ultraviolet (UV) light, and particularly with the use of UV-C light (i.e. light having a wavelength of between about 180 nm and about 280 nm, inclusive). The apparatuses can use light emitting diodes (LEDs) as a source of UV-C light. The LEDs can be high-efficiency LEDs.

In some examples, the apparatuses have a “pass-through” configuration, in which objects enter the apparatus from one side (a “dirty side”) and exit on another side (a “clean side”). Such apparatuses may be particularly useful in hospital or long-term care settings, or other settings that require distancing of contaminated objects from “clean” areas (although the apparatuses are not limited to use in such settings). For example, “pass-through” apparatuses can optionally be mounted in a wall separating a “clean” room (e.g. a hospital room housing an immune-compromised patient) from the exterior environment (e.g. the hallway of a hospital), so that objects can be passed into the clean room via the apparatus and decontaminated prior to entering the clean room.

In some examples, the apparatuses include a belt conveyor system for automatically conveying objects through the device. This can allow for high throughput.

The apparatuses can include various safety features. For example, the apparatuses can allow users to view the contents thereof during a decontamination cycle, while preventing UV-C light from exiting the apparatuses. For further example, the apparatuses can be configured to prevent opening thereof during a decontamination cycle.

The apparatuses can optionally use low-voltage LEDs, and can include a power adapter for connection to a power source. This can allow for the apparatuses to be used in various settings. For example, by using low-voltage LEDs, the apparatuses can be powered by various sources such as batteries or generators, and can be used in a field-hospital, a remote setting, and/or in disaster relief.

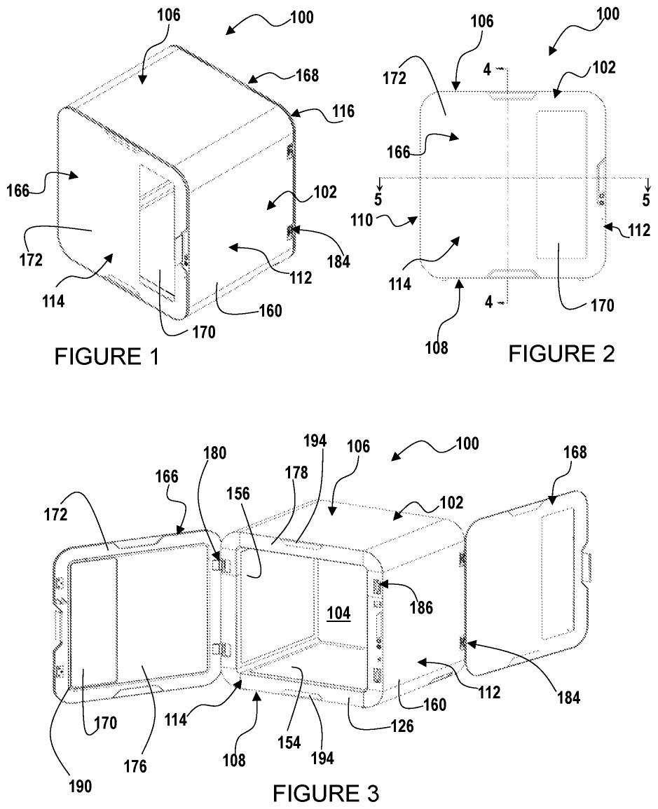

Referring now to FIGS. 1 to 3, a first example of a decontamination apparatus 100 (also referred to herein simply as an ‘apparatus’) is shown. The decontamination apparatus 100 includes a housing 102, which defines an interior volume 104 (visible in FIG. 3). Various objects (examples of which are mentioned above) may be placed in the interior volume 104 for decontamination.

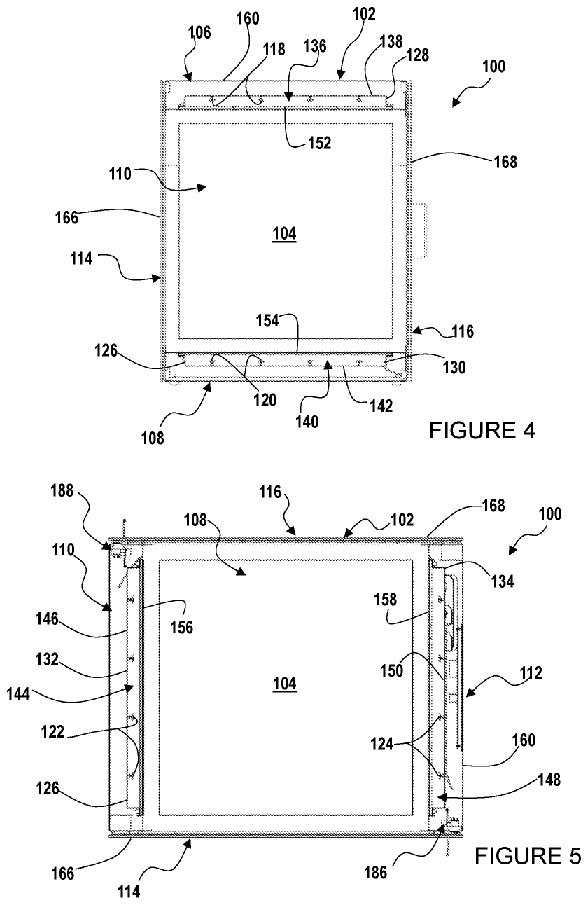

Referring still to FIGS. 1 to 3, the housing 102 generally has a top 106, a bottom 108 (labelled only in FIGS. 2 and 3), a left side 110 (labelled only in FIG. 2), a right side 112, a front 114, and a rear 116 (labelled only in FIG. 1). The housing 102 can be configured so that top 106, the bottom 108, the left side 110, the right side 112, the front 114, and/or the rear 116 includes a UV-C light source that faces inwardly towards the interior volume 104. As used herein, the term “faces” indicates that the light source is positioned to emit light to the interior volume, either directly (i.e. by travelling in a straight line towards the interior volume), or indirectly (e.g. by reflecting off a surface before reaching the interior volume). In the example shown, the decontamination apparatus 100 is configured so that UV-C light is emitted to the interior volume 104 from the top 106, the bottom 108, the left side 110, and the right side 112. More specifically, referring to FIG. 4, the top 106 includes a set of UV-C LEDs 118 (only two of which are labelled) that face towards the interior volume 104, and the bottom 108 includes a set of UV-C LEDs 120 (only two of which are labelled) that face towards the interior volume 104. Referring to FIG. 5, the left side 110 includes a set of UV-C LEDs 122 (only two of which are labelled) that face towards the interior volume 104, and the right side 112 includes a set of UV-C LEDs 124 (only two of which are labelled) that face towards the interior volume 104.

As mentioned above, the term UV-C LED refers to an LED that emits light having a wavelength of between about 180 nm and about 290 nm. For example, the light can have a wavelength of between about 254 and about 285 nm. Alternatively, the light can be in the far UV-C range, i.e. between about 200 nm and about 220 nm. The UV-C LEDs can be monochromatic or can be broad-spectrum. The UV-C LEDs can all emit light of the same peak wavelength, or can emit light of different peak wavelengths.

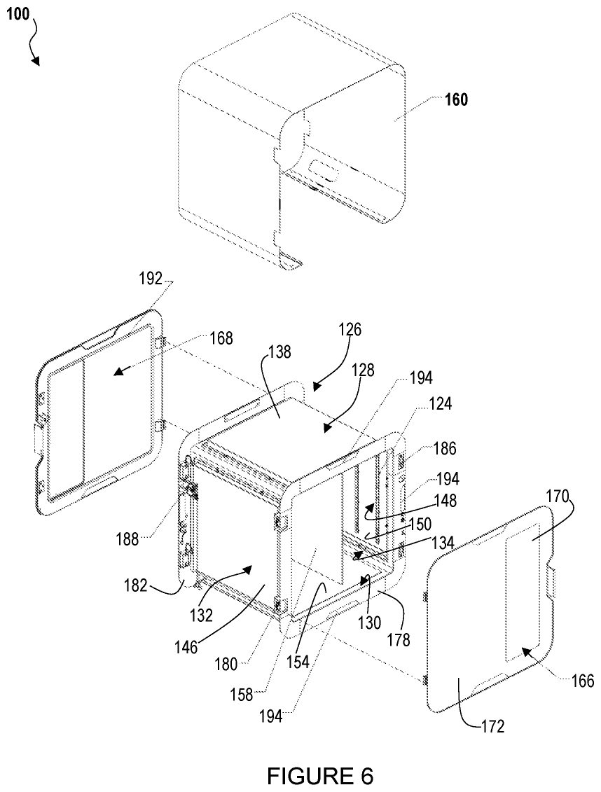

In the example shown, the UV-C LEDs 118, 120, 122, 124 are provided by UV-C LED strips (visible in FIG. 6), which position the UV-C LEDs in a grid. In alternative examples, the UV-C LEDs can be provided as individual bulbs, and can be arranged in a fashion other than a grid. Each set of UV-C LEDs can include, for example, 72 or more UV-C LEDs, or between 72 and 120 UV-C LEDs. For example, the top 106 can include 72 UV-C LEDs, which can be provided by 4 strips of 18 UV-C LEDs. The bottom 108, the left side 110, and the right side 112 can be of a similar configuration.

In some examples, the UV-C LEDs 118, 120, 122, 124 are relatively small—e.g. they can each have a depth of about ¼ inch or less.

The UV-C LEDs 118, 120, 122, 124 can be low voltage LEDs. For example, the UV-C LEDs 118, 120, 122, 124 can have a voltage of between about 10V DC and about 60V DC, inclusive. In some examples, the UV-C LEDs 118, 120, 122, 124 can have a voltage of about 24V DC. A power adaptor (not shown) can be provided for connecting the low-voltage UV-C LEDs 118, 120, 122, 124 to a variety of power sources, such as a 120V socket, or a 240V socket, or a 220 volt socket, or a mobile generator, or a battery. As mentioned above, this can allow for the decontamination apparatus 100 to be used in various settings.

Referring now to FIG. 6, in the example shown, the apparatus 100 includes an inner shell 126. The inner shell 126 provides an inner wall 128 of the top 106, an inner wall 130 of the bottom 108, an inner wall 132 of the left side 110, and an inner wall 134 of the right side 112. Referring also to FIG. 4, the inner wall 128 defines a recess 136 in which the set of UV-C LEDs 118 are nested, and includes a support panel 138 for the set of UV-C LEDs 118. The set of UV-C LEDs 118 are mounted to the support panel 138. Likewise, the inner wall 130 defines a recess 140 in which the set of UV-C LEDs 120 are nested, and includes a support panel 142 for the set of UV-C LEDs 120. Referring also to FIG. 5, the inner wall 132 defines a recess 144 in which the set of UV-C LEDs 122 are nested, and includes a support panel 146 for the set of UV-C LEDs 122. Likewise, the inner wall 134 defines a recess 148 in which the set UV-C LEDs 124 are nested, and includes a support panel 150 for the set of UV-C LEDs 124. In the example shown, the inner walls 128, 130, 132, and 134 are UV-C blocking. That is, UV-C rays cannot pass through the inner walls 128, 130, 132, and 134 in any significant amount (e.g. in an amount that can physically harm a person, that is detectable with standard test equipment, and that exceeds accepted safety standards). Example materials suitable for use in the inner walls 128, 130, 132, and 134 are metals such as stainless steel, acrylic plastic, or a specialty glass or derivative.

Referring to FIG. 4, the top 106 further includes a UV-C transmissive panel 152 that is mounted over the recess 136, between the set of UV-C LEDs 118 and the interior volume 104. The UV-C transmissive panel 152 is configured to allow the passage of all or a majority of the UV-C light coming from the UV-C LEDs 118. Example materials suitable for use in the UV-C transmissive panels are silica glass (e.g. high-purity silica glass), quartz glass (e.g. high-purity quartz glass), and UV-C transmissive plastics (e.g. an acrylic). Likewise, the bottom 108 includes a UV-C transmissive panel 154 that is mounted over the recess 140, between the set of UV-C LEDs 120 and the interior volume 104. Referring to FIG. 5, the left side 110 includes a UV-C transmissive panel 156 that is mounted over the recess 144, between the set of UV-C LEDs 122 and the interior volume 104, and the right side 112 includes a UV-C transmissive panel 158 that is mounted over the recess 148, between the set of UV-C LEDs 124 and the interior volume 104.

In alternative examples, one or more of the UV-C transmissive panels can be fabricated from another material, such as a UV-C reflective material that has perforations extending therethrough to allow for the transmission of UV-C light. One example of such a material is perforated polytetrafluoroethylene (PTFE).

Referring to FIGS. 4 to 6, the apparatus 100 further includes an outer skin 160 that clads the inner walls 128, 132, and 134. The outer skin 160 can be fabricated from, for example, metals or plastics.

Referring to FIGS. 1, 3, and 6, in the example shown, the apparatus 100 is of a “pass-through” configuration. That is, in the example shown, the front 114 includes a front opening, and the rear 116 includes a rear opening, and objects can be inserted into the interior volume 104 via the front opening, and removed from the interior volume 104 via the rear opening (or vice versa). The front 114 further includes a front door 166 that is openable (as shown in FIG. 3) and closable (as shown in FIG. 1) to block the front opening, and the rear 116 includes a rear door 168 that is openable (as shown in FIG. 3) and closable (as shown in FIG. 1) to block the rear opening.

Referring still to FIGS. 1, 3, and 6, in the example shown, the front door 166 is configured to provide a viewing window 170, while preventing UV-C rays from passing therethrough, and while reflecting at least some (e.g. all or a majority) of the UV-C rays hitting the front door 166 back into the interior volume 104. In the example shown, the front door 166 includes a transparent panel 172 that is UV-C blocking. For example, the transparent panel 172 can be a low-emissivity glass panel or a composite panel. The front door 166 has an inner surface that faces towards the interior volume 104, and the inner surface is UV-C reflective, so that UV-C rays emitted from the UV-C LEDs 118, 120,122, 124 are reflected by the inner surface back towards the interior volume 104. In the example shown, the UV-C reflective inner surface is provided by a mirror-finish stainless steel lining 176 (shown in FIG. 3) on the transparent panel 172. In alternative examples, the UVC-reflective surface can be provided by a PTFE lining on the transparent panel 172, or anodized aluminum. The lining 176 has a cut-out therein, which provides the front door with the viewing window 170 (which is UV-C blocking). The viewing window 170 can be relatively small (e.g. in the example shown takes up less than half of the area of the front door 166), to still allow a majority of the UV-C rays hitting the front door 166 to be reflected by the inner surface. The rear door 168 is of the same configuration as the front door 166, and for brevity, is not described in detail herein.

Referring to FIGS. 3 and 6, the inner shell 126 includes a front flange 178, and the front door 166 is mounted to the front flange 178 by a pair of hinges 180 (only one of which is labelled) along the left side 110. The inner shell 126 includes a rear flange 182 (shown only in FIG. 6), and the rear door 168 is mounted to the rear flange 182 by a pair of hinges 184 (shown only in FIG. 3, only one of which is labelled) along the right side 112. For safety, in order to prevent opening of the front 166 and rear 168 doors during illumination of the UV-C LEDs 118, 120,122, 124 (i.e. during a decontamination cycle), a front locking mechanism 186 is provided for the front door 166 and a rear locking mechanism 188 (shown only in FIG. 6) is provided for the rear door 168. The locking mechanisms 186, 188 can include, for example, magnetic locks that are configured to prevent opening of the front 166 and rear 168 doors during illumination of the UV-C LEDs 118, 120, 122, 124. Furthermore, for safety, the front door 166 includes a UV-C blocking gasket 190 (shown only in FIG. 3) that prevents escape of UV-C rays between the inner shell 126 and the front door 166 when the front door 166 is closed. Similarly, the rear door 168 includes a UV-C blocking gasket 192 (shown only in FIG. 6) that prevents escape of UV-C rays between the inner shell 126 and the rear door 168 when the rear door 168 is closed. The UV-C blocking gaskets can be made from, for example, a foam such as an ethylene propylene diene monomer foam.

Referring still to FIGS. 3 and 6, in the example shown, the apparatus 100 further includes a set of indicators 194 (only some of which are labelled), which can illuminate to indicate a status of the apparatus 100.

The apparatus 100 can be of various sizes. For example, the apparatus 100 can be about 19 inches in height, 16 inches in depth, and 19 inches in width, or about 33 inches in height, 28 inches in depth, and 33 in width, or about 60 inches in height, 54 inches in depth, and 60 in width. In some examples, the apparatus 100 can be sized for a person to walk through, while wearing protective clothing to prevent physical personal harm. For example, in a hospital setting, a person wearing personal protective equipment that is UV-C blocking (e.g. a UV-C blocking hazmat suit) can walk through the apparatus 100 in order to decontaminate the personal protective equipment. In such examples, the apparatus 100 can be, for example, about 108 inches in height, 53 inches in depth, and 60 inches in width.

As mentioned above, the apparatus 100 can be mounted in a wall separating a “clean room” from the exterior environment, so that objects can be passed into the clean room via the apparatus 100 and decontaminated prior to entering the clean room. Alternatively, the apparatus 100 can be a desktop apparatus, or can be free-standing, or can be a mobile apparatus (e.g. it can be provided on a cart).

In use, an object to be decontaminated (examples of which are listed above) can be placed in the interior volume 104 via the front 166 or rear 168 door, and can rest on the UV-C transmissive panel 154 of the bottom 108. With the front 166 and rear 168 doors closed, a decontamination cycle can then be started, for example by pushing a start button (not shown) of the apparatus 100, and/or by setting a timer (not shown) of the apparatus 100, which can cause the front 166 and rear 168 doors to lock and the UV-C LEDs 118, 120,122, 124 to illuminate. A decontamination cycle can last, for example, for less than about 5 minutes (e.g. for about 1 minute, or for about 30 seconds). In this time period, UV-C rays decontaminate the object by hitting the object directly from the top 106, the bottom 108, the left side 110, and the right side 112 of the apparatus 100, and hitting the object by being reflected from by front 166 and rear 168 doors. Upon completion of the decontamination cycle, the UV-C LEDs 118, 120, 122, 124 can turn off (e.g. automatically at the end of a set time, or manually by pushing a stop button), and the front 166 and rear 168 doors can unlock. The object can then be removed from the apparatus 100 via the front door 166 or the rear door 168.

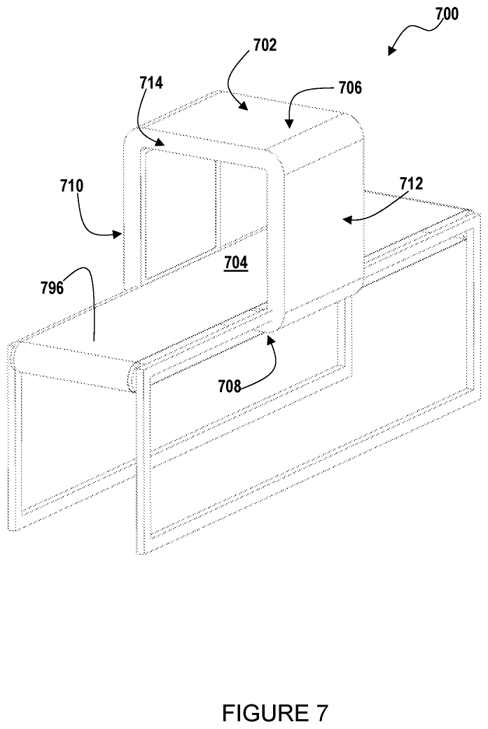

Referring now to FIG. 7, another example of a decontamination apparatus is shown. In FIG. 7, features that are like those of FIGS. 1 to 6 will be referred to with like reference numerals, incremented by 600.

The decontamination apparatus 700 of FIG. 7 is similar to the decontamination apparatus 100 of FIGS. 1 to 6, and includes a housing 702 having an interior volume 704 and top 706, a bottom 708, a left side 710, a right side 712, a front 714, and a rear (not visible in FIG. 7). The top 706, the bottom 708, the left side 710, and the right side 712 each include a UV-C light source (not shown, which can be in the form of UV-C LEDs as described above) that faces inwardly towards the interior volume 704. The front 714 includes a front opening and the rear includes a rear opening.

Unlike the apparatus 100 of FIGS. 1 to 6, the front 714 and rear do not include doors, and a belt conveyor system passes through the interior volume 704 from outboard of the front opening to outboard of the rear opening, to allow objects to be conveyed through the interior volume 704. In the example shown, the belt conveyor system includes a conveyor belt 796 and at least two pulleys (not shown). The size of the housing 702 and the speed of the conveyor belt 796 can be tuned so that the time it takes an object to pass through the housing 702 is sufficient for decontamination.

For safety, the apparatus 700 can include UV-C blocking covers (not shown) that cover the front and rear openings while still allowing objects to enter and exit via the front and rear openings. For example, the apparatus 700 can include a front UV-C blocking curtain (not shown) covering the front opening and a rear UV-C blocking curtain (not shown) covering the rear opening. The UV-C blocking curtains can optionally be configured to reflect UV-C light back towards the interior volume 704. Furthermore, the UV-C light source can be angled such that UV-C rays are not directed out of the front and rear openings, or such that the quantity of UV-C rays exiting the front and rear openings is minimized.

In the example shown, as UV-C light is emitted from the bottom 708, the conveyor belt 796 is UV-C transmissive, in order to allow the UV-C rays from the bottom 708 to hit objects on the conveyor belt 796. In alternative examples, UV-C light can be emitted from only the top 706, the left side 710, and the right side 712, and the belt-conveyor system can further include a flipping apparatus (not shown) for flipping objects on the conveyor belt 796, to allow all surfaces of the object to be hit with UV-C rays.

Similarly to the apparatus 100 of FIGS. 1 to 6, the apparatus 700 of FIG. 7 can be mounted in a wall, or can be free-standing, or can be a desktop apparatus, or can be a mobile apparatus.

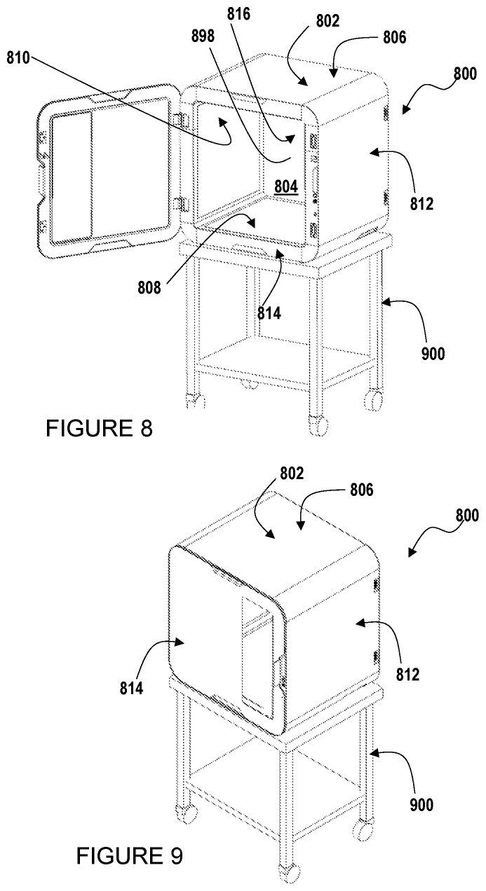

Referring now to FIGS. 8 and 9, another example of a decontamination apparatus is shown. In FIGS. 8 and 9, features that are like those of FIGS. 1 to 6 will be referred to with like reference numerals, incremented by 800.

The decontamination apparatus 800 of FIGS. 8 and 9 is similar to the decontamination apparatus 100 of FIGS. 1 to 6, and includes a housing 802 having an interior volume 804 and a top 806, a bottom 808 (visible only in FIG. 8), a left side 810 (visible only in FIG. 8), a right side 812, a front 814, and a rear 816 (visible only in FIG. 8). The top 806, the bottom 808, the left side 810, and the right side 812 each include a UV-C light source (not shown, which can be in the form of UV-C LEDs as described above) that faces inwardly towards the interior volume 804. Unlike the apparatus 100 of FIGS. 1 to 6, the rear 816 does not include a rear door. Instead, the rear 816 is closed by a rear wall 898 (visible only in FIG. 8) of the housing 802. In the example shown, the rear wall 898 includes an inner surface that is UV-C reflective, to reflect UV-C rays that are emitted from the top 806, the bottom 808, the left side 810, and the right side 812. In alternative examples, the rear 816 can be configured to include a set of UV-C LEDs

The apparatus 800 of FIG. 8 can be, for example, mounted in a wall, free-standing, a desktop apparatus, or a mobile apparatus (e.g. it can sit on a cart 900, as shown).

In any of the above examples, the apparatus can include interior shelves (such as UV-C transmissive shelves) and/or interior hangers.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A decontamination apparatus comprising:
a housing defining an interior volume and having at least a top, a bottom, a left side, a right side, a front, and a rear;
wherein at least one of the top, the bottom, the left side, and the right side comprises a UV-C light source that faces inwardly towards the interior volume; and
wherein the front comprises a front opening and a front door that is openable and closable to block the front opening, and the rear comprises a rear opening and a rear door that is openable and closable to block the rear opening, to allow objects to pass through the interior volume from the front to the rear.

2. The decontamination apparatus of claim 1, wherein at least one of the front door and the rear door comprises a UV-C blocking viewing window.

3. The decontamination apparatus of claim 2, wherein the UV-C blocking viewing window comprises a low-emissivity glass.

4. The decontamination apparatus of claim 1, wherein the front door and the rear door each have, respectively, a UV-C reflective inner surface that faces the interior volume.

5. The decontamination apparatus of claim 4, wherein the UV-C reflective inner surfaces comprise mirror-finish stainless steel, anodized aluminum or polytetrafluoroethylene.

6. The decontamination apparatus of claim 1, wherein the front door and the rear door are lockable to prevent opening during illumination of the UV-C light source.

7. The decontamination apparatus of claim 1, wherein the front door and the rear door each comprise, respectively, a UV-C blocking gasket for preventing escape of UV-C light when the front door and rear door are closed.

8. The decontamination apparatus of claim 1, wherein each of the top, the bottom, the left side, and the right side comprises, respectively, a set of UV-C light emitting diodes (LEDs) that face towards the interior volume.

9. The decontamination apparatus of claim 8, wherein the UV-C LEDs have a depth that is equal to or less than ¼ inch.

10. The decontamination apparatus of claim 8, wherein each set comprises at least 72 of the UV-C LEDs.

11. The decontamination apparatus of claim 8, wherein the UV-C LEDs are low voltage, and the decontamination apparatus further comprises a power adaptor for connecting the UV-C LEDs to a power source.

12. The decontamination apparatus of claim 11, wherein the voltage of the UV-C LEDs is between 10V DC and 60V DC.

13. The decontamination apparatus of claim 11, wherein the voltage of the UV-C LEDs is about 24 V DC.

14. The decontamination apparatus of claim 8, wherein each of the top, the bottom, the left side, and the right side comprises, respectively, a UV-C transmissive panel between the UV-C LEDs and the interior volume.

15. The decontamination apparatus of claim 14, wherein the UV-C transmissive panels comprise silica glass, an acrylic, or perforated polytetrafluoroethylene.

16. The decontamination apparatus of claim 8, wherein each of the top, the bottom, the left side, and the right side comprises, respectively, an inner wall comprising a support panel that supports the set of UV-C LEDs and that defines a recess in which the UV-C LEDs are nested.

17. The decontamination apparatus of claim 16, wherein the support panels are UV-C blocking.

18. The decontamination apparatus of claim 16, wherein the support panels comprise a metal.
